(19) United States
(12) Patent Application Publication
(10) Pub. No.: US 2025/0099038 A1
(43) Pub. Date: Mar. 27, 2025

(54) DEVICES, SYSTEMS, AND METHODS FOR CONTROLLING IMPLANTABLE SENSORS

(71) Applicant: OrthoSensor, Inc., Dania Beach, FL (US)

(72) Inventors: Andrew MEYER, Davie, FL (US); Matthias VERSTRAETE, Chaam (NL)

(73) Assignee: OrthoSensor, Inc., Dania Beach, FL (US)

(21) Appl. No.: 18/473,817

(22) Filed: Sep. 25, 2023

(51) Int. Cl. A61B 5/00; A61B 5/11

(52) U.S. Cl. CPC A61B 5/686; A61B 5/112; A61B 5/4851; A61B 2560/0209

(57) ABSTRACT

The present disclosure generally relates to a surgical implant comprising: an accelerometer; a sensor; and a processor. The processor configured to: receive a first data from the accelerometer based on a movement of the surgical implant; determine a type of movement of the surgical implant based on the first data; and adjust power to the sensor based on the type of movement of the surgical implant.

### TECHNICAL FIELD

The present disclosure relates generally to devices, systems, and methods of controlling implantable sensors, wearable sensors, implant devices, prosthetics, and other medical devices.

### BACKGROUND

The skeletal system of a mammal is subject to variations among species. Further changes may occur due to environmental factors, injury, degradation through use, and aging. An orthopedic joint of the skeletal system typically comprises two or more bones that move in relation to one another. Movement is enabled by muscle tissue and tendons attached to the skeletal system of the joint. Ligaments hold and stabilize the one or more joint bones. Cartilage is a wear surface that prevents bone-to-bone contact, distributes load, and lowers friction.

There have been substantial developments in the repair of the human skeletal system. In general, orthopedic joints have evolved using information from simulations, mechanical prototypes, and patient data that is collected and used to initiate improved designs. Similarly, the tools being used for orthopedic surgery have been refined over the years.

Each joint replacement procedure is subject to significant variation from patient to patient. Further, once a patient has had a joint partially or totally replaced, rehabilitation and returning to activities may be challenging. There is a need for systems, methods, and devices to facilitate patient rehabilitation from orthopedic surgery, such as joint replacement surgery.

### SUMMARY

The present disclosure generally relates to a surgical implant comprising: an accelerometer; a sensor; and a processor. The processor may be configured to: receive a first data from the accelerometer based on a movement of the surgical implant; determine a type of movement of the surgical implant based on the first data; and adjust power to the sensor based on the type of movement of the surgical implant.

Further aspects of the present disclosure provide: wherein the sensor is powered off during a first movement; sensor is powered on during a second movement; wherein sensor processing is off during a first movement; wherein sensor processing is on during a second movement; wherein the sensor is multiple sensors; wherein the sensor is at least one of a temperature sensor, a load sensor, and a gyroscope; wherein in a first movement the sensor is deactivated and processor does not process; wherein in a second movement the sensor is activated; wherein the determined type of movement is walking; wherein the determined type of movement is at least one of ascending stairs, descending stairs, changing position between horizontal and vertical, and aerobic activity; wherein the surgical implant is a knee implant; wherein the surgical implant is a shoulder implant; wherein the surgical implant is a spine implant.

The present disclosure further relates to a method for measuring movement of an implant via a measurement device, the method including: receiving, via at least one processor, first data from a first sensor, wherein: the measurement device is coupled to a musculoskeletal system of a patient; determining a patient movement based on the first data; and adjusting the power supplied to a second sensor based on determined patient movement.

Further aspects of the present disclosure include: further comprising initiating collection of second data from the second sensor based on the determined patient movement; wherein the second data is communicated with one or more remote systems; further comprising activating the second sensor based on the determined patient movement; wherein the determined patient movement is a first determined patient movement, the method further comprising determining a second patient movement based on the first data and the second data; wherein the first data and the second data are used to update patient movement identification; wherein determining a patient movement based on the first data includes determining the patient movement based on the first data and a third data from a database; wherein the database includes data from multiple users; wherein the database includes data collected from one or more prior patients with one or more features in common; wherein the first data is received from an accelerometer; wherein machine learning is used to determine the patient movement; wherein an algorithm of the machine learning is adjusted from one or more prior patient data sets; wherein the database includes accelerometer data associated with one or more prior patients with one or more features in common with the patient.

The present disclosure further relates to a method of determining a movement of a patient, the method including: determining a threshold sensor value of a first sensor associated with a first movement type using one or more prior patient datasets; receiving a first data from a first sensor of an implantable measurement device; and determining a new threshold sensor value using one or more prior patient data sets and the first data.

Further aspects of the present disclosure include: further comprising: activating a second sensor when a first sensor value of the first sensor exceeds the new threshold sensor value; wherein the implantable measurement device includes a battery, and the method further comprising: monitoring a power level of the implantable measurement device; and further comprising adjusting a sample rate of the first sensor.

### BRIEF DESCRIPTION OF THE FIGURES

The accompanying drawings, which are incorporated in and constitute a part of this specification, illustrate various exemplary aspects of the disclosure, and together with the description serve to explain the principles of the present disclosure.

FIG. 1 depicts an exploded view of an implantable measurement device configured as a prosthetic knee joint system in accordance with an example embodiment.

FIG. 2 illustrates an exploded view of an implantable measurement device in accordance with an example embodiment.

FIG. 3 illustrates a schematic diagram of a data collection system including example devices connected with a communication network, in accordance with an exemplary embodiment.

FIG. 4 illustrates an example of an implantable measuring device incorporated with a ball-and-socket joint, in accordance with an exemplary embodiment.

FIG. 5 illustrates an example of an implantable measuring device incorporated with a ball-and-socket joint, in accordance with an exemplary embodiment.

FIG. 6 illustrates an example of an implantable measuring device configured as a screw, in accordance with an exemplary embodiment.

FIG. 7 illustrates a block diagram of a control strategy for an implantable measuring device, accordingly to aspects of this disclosure.

FIG. 8 illustrates a block diagram of a control strategy for an implantable measuring device, accordingly to aspects of this disclosure.

### DETAILED DESCRIPTION

Embodiments of this disclosure are broadly directed to measurement of physical parameters, and more particularly to devices, systems, and methods for orthopedic correction and data collection. Various aspects of this disclosure may improve measurement accuracy, improve surgical outcomes, and/or reduce cost or time in surgery and/or rehabilitation. The following description of exemplary embodiments is merely illustrative in nature and is in no way intended to limit the invention, its application, or uses. Particular aspects of this disclosure are described in greater detail below. The terms and definitions provided herein control, if in conflict with terms and/or definitions incorporated by reference.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.” As used herein, the singular forms “a,” “an,” and “the” include plural reference unless the context dictates otherwise. The term “or” is used disjunctively, and thus a list set off by the term “or” may include any number of any of the items in the list. As used herein, the terms “approximately” and “about” should be understood to encompass±10% of a specified amount or value (e.g., “about 90%” may refer to the range of values from 81% to 99%).

For simplicity and clarity of the illustrations, elements in the figures are not necessarily to scale, are only schematic, are non-limiting, and the same reference numbers in different figures denote the same elements, unless stated otherwise. Additionally, descriptions and details of well-known steps and elements are omitted for simplicity of the description. Notice that once an item is defined in one figure, it may not be discussed or further defined in the following figures.

The terms “first”, “second”, “third” and the like in the Claims or/and in the Detailed Description are used for distinguishing between similar elements and not necessarily for describing a sequence, either temporally, spatially, in ranking or in any other manner. It is to be understood that the terms so used are interchangeable under appropriate circumstances and that the embodiments described herein are capable of operation in other sequences than described or illustrated herein.

Processes, techniques, apparatus, and materials as known by one of ordinary skill in the art may not be discussed in detail but are intended to be part of the enabling description where appropriate.

The orientation of the X, Y, and Z-axes of rectangular Cartesian coordinates is assumed to be such that the X and Y-axes define a plane at a given location, and the Z-axis is normal to the X-Y plane. The axes of rotations about the Cartesian axes of the device are defined as yaw, pitch and roll. With the orientation of the Cartesian coordinates defined in this paragraph, the yaw axis of rotation is the Z-axis through body of the device. Pitch changes the orientation of a longitudinal axis of the device. Roll is rotation about the longitudinal axis of the device.

The orientation of the X, Y, Z-axes of rectangular Cartesian coordinates is selected to facilitate graphical display on computer screens having the orientation that the user will be able to relate to most easily. Therefore, the image of the device moves upward on the computer display whenever the device itself moves upward for example away from the surface of the earth. The same applies to movements to the left or right.

The terms ‘motion sensing’, ‘tilt sensing’, and ‘orientation’ are also intended to have specific meaning. ‘Motion sensing’ generally encompasses the detection of movement of a body that exceeds a specified threshold in one or more coordinate axes, for example the specific threshold in one or more Cartesian axes in terms of both static and dynamic acceleration. Further, “threshold” may be one or more values of one or more different characteristics measured by a sensor, determined by a control system, processed by a processor, user defined, or a combination thereof. In some examples, a threshold may be a first value relating to a first characteristic (e.g. velocity along the x-axis), and a second value relating to a second characteristic (e.g. velocity along the y-axis), such that to reach the threshold, both the first value and the second value must be met In another example, a threshold may be a first value relating to a first characteristic (e.g. velocity along x-axis, y-axis, and/or z-axis), and a second value relating to a second characteristic (e.g. angle relative to earth's gravity vector), such that to reach the threshold, both the first value and the second value must be met. In some examples, a threshold may be a first value relating to a first characteristic (e.g. velocity along x-axis, y-axis, z-axis, or a combination thereof), and a second value relating to a second characteristic (e.g. frequency of movement), such that to reach the threshold, both the first value and the second value must be met. In some other examples, a threshold may be a combination of two or more, three or more, four or more, or even a plurality of different values associated with different characteristics. ‘Heading’ generally encompasses the orientation of longitudinal axis of the motion and orientation sensing module or device and movement in a direction. ‘Tilt’ generally encompasses the orientation of a body with respect to a vertical axis. The term slope is used interchangeable with the term “tilt.” ‘Tilt sensing’ generally encompasses the measurement of acceleration attributable to gravity in one or more axes. ‘Orientation’ generally encompasses yaw as well as ‘tilt.’ Note that although accelerometers and gyroscopes are provided as enabling examples in the description of embodiments, any tracking device (e.g., a GPS chip, acoustical ranging, magnetometer, inclinometers, hybrid sensors, MEMs) may be used within the scope of the embodiments described.

In general, kinetics is the study of the effect of forces upon the motion of a body or system of bodies. Disclosed herein is a system for kinetic assessment and measurement of the muscular-skeletal system. The kinetic system may be for the installation of prosthetic components or for monitoring, measuring, and assessment of permanently installed components to the muscular-skeletal system. For example, installation of a prosthetic component may require one or more bone surfaces to be prepared to receive a device or component. The bone surfaces are cut to place the prosthetic component in a relational position to a mechanical axis of a joint. The kinetic system is designed to take quantitative measurements of at least the load, position of load, and alignment with the forces being applied to the joint similar to that of a final joint installation. The sensored measurement components are designed to allow ligaments, tissue, and bone to be in place while the quantitative measurement data is taken. This is significant because the bone cuts take into account the kinetic forces where a kinematic assessment and subsequent bone cuts could be substantial changed from an alignment, load, and position of load once the joint is reassembled.

In some examples, sensors in the prosthetic components may provide periodic data related to the status of the implant in use. Data collected intra-operatively and long term (post-operatively) may be used to facilitate rehabilitation, determine parameter ranges for surgical installation, post-operative recovery, post-operative activity, and to improve future prosthetic components. Often, several measured parameters or different measurements may be used to make a quantitative assessment. Parameters may be evaluated relative to orientation, alignment, direction, displacement, or position as well as movement, rotation, or acceleration along an axis or combination of axes by wireless sensing modules or devices positioned on or within a body, instrument, appliance, vehicle, equipment, or other physical system. In some examples, a graphical user interface may support assimilation of measurement data. Parameters may be evaluated relative to orientation, alignment, direction, displacement, or position as well as movement, rotation, or acceleration along an axis or combination of axes by wireless sensing modules or devices positioned on or within a body, instrument, appliance, vehicle, equipment, or other physical system.

The kinetic system is designed to take quantitative measurements of one or more of a load, position of load, and alignment with the forces being applied to the joint similar, as well as, the amount of movement within the joint. Further, the kinetic system may be designed to monitor the type of movement which a patient is performing based on at least the load, position of load, alignment, and/or amount of movement of the joint.

The example embodiments shown herein below of a measurement device are illustrative only and do not limit use for other parts of a body. The measurement device may be a tool, equipment, implant, insert, or prosthesis that measures at least one parameter or supports installation of prosthetic components to the musculoskeletal system. The measurement device may be used on bone, the knee, hip, ankle, spine, shoulder, hand, wrist, foot, fingers, toes, and other areas of the musculoskeletal system. In general, the principles disclosed herein are meant to be adapted for use in all locations of the musculoskeletal system.

The following examples are generally provided to illustrate several exemplary control strategies implemented onto a prosthetic component of the kinetic system configured as an implantable measurement device. More particularly, the control strategies may be directed to selectively activating and deactivating one or more sensors, one or more processes, or both, of the implantable measurement device. Additionally, the example control strategies may be directed to selectively controlling a sampling rate of the one or more sensors of the implantable measurement device (e.g., the number of cycles which a sensor may actively record data).

Generally, the implantable measurement device may be configured to determine a target activity from a non-target activity, so that data associated with the target activity may be recorded and analyzed. A target activity may be an activity that is selected to be monitored and analyzed. The type of implantable measurement device that is implanted into a patient and a patient's particular diagnosis and treatment may determine the type of activity that is considered a target activity. In some examples, when the implantable measurement device is configured as a knee implant, the target activity may be running, ascending stairs, descending stairs, changing position from seated to standing, changing position from standing to seated, changing between horizontal (e.g. lying down) and vertical (e.g. standing) positions, performing aerobic activity, riding a bike, participating in a sporting activity (e.g., playing tennis), the like, or a combination thereof. Similarly, when the implantable measurement device is configured as a hip implant, the target activity may be running, climbing stairs, changing position from seated to standing, changing position from standing to seated, riding a bike, participating in a sporting activity (e.g., playing tennis), the like, or a combination thereof. In another example, when the implantable measurement device is configured as a shoulder implant, a target activity may be over-head reaching, lifting arm(s) past a threshold angle, participating in a sporting activity (e.g., playing tennis), the like, or a combination thereof. A non-target activity may be an activity that does not require activity data collection since the activity puts relatively little strain on the patient (e.g., walking). Any of the described target activities may be monitored in any of the measurement devices described herein.

Collecting target activity data may be used to improve post-operative care and patient quality scores. By collecting data relating to target activities, a physician and/or physical therapist may help improve a patient's post-operative experience and rehabilitation.

### System Overview

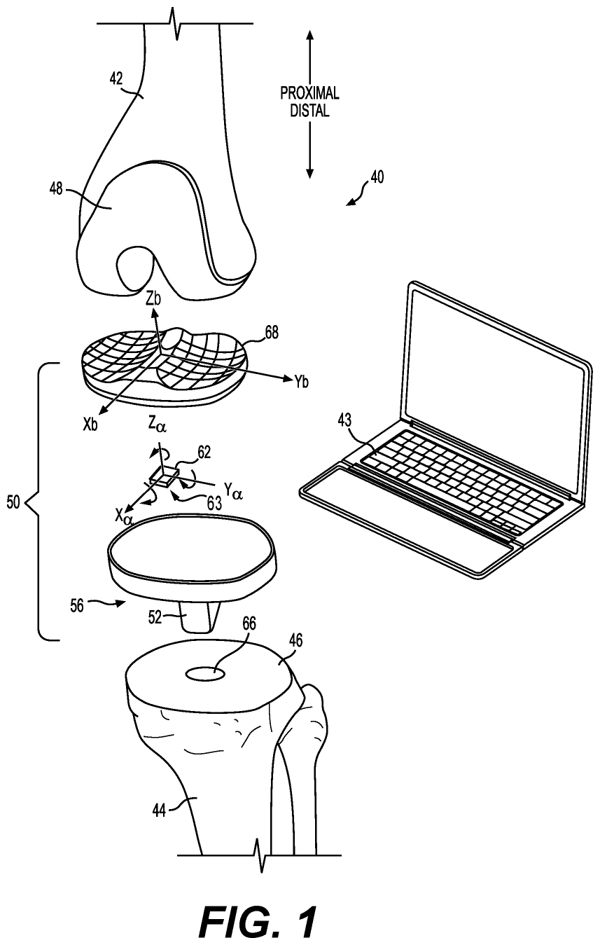

FIG. 1 is an exploded view of a prosthetic knee joint system 40 including an implantable measurement device 50 in accordance with an example embodiment configured to measure one or more characteristics of a leg and/or a joint. In this example, the prosthetic knee joint 40 may comprise a prosthetic component 48 coupled to a distal end of a femur 42, and an implantable measurement device 50 coupled to a proximal end of a tibia 44. In some examples, such as shown in FIG. 1, implantable measurement device 50 may include an inertial measurement unit 62 (also referred to as an IMU). The inertial measurement unit 62 may be operatively coupled to the knee joint, to support real-time activity measurement of the knee. Implantable measurement device 50 may include support 68, inertial measurement unit 62, and base 56. In some examples, the inertial measurement unit 62 is located between support 68 and base 56 of implantable measurement device 50. In one example, inertial measurement unit 62 may be operatively coupled to tibia 44. For example, inertial measurement unit 62 may be positioned within tibial stem or keel 52 of implantable measurement device 50. Although not shown in the figures, it is contemplated that an inertial measurement unit may be operatively connected with an implant which is connected with femur 42.

Inertial measurement unit 62 may comprise a plurality of sensors, such as one or more gyroscopes, one or more accelerometers, other sensors described further below, and the like. In some examples, the inertial measurement unit 62 may include three gyroscopes, three accelerometers, or both. It is contemplated that any number of gyroscopes, accelerometers, or other sensors may be incorporated into the inertial measurement unit 62. In one example, a first, second, and third gyroscope and a first, second, and third accelerometer may respectively be aligned to Xa, Ya, and Za axes. Each gyroscope may measure an angular velocity corresponding to rotation about an axis as indicated on the diagram. Each accelerometer, similarly, may measure a change in motion (acceleration) corresponding to one of the axes.

With continued reference to FIG. 1, the proximal end of tibia 44 has a prepared bone surface 46. Prepared bone surface 46 of tibia 44 is cut relative to a reference. For example, the proximal end of tibia 44 may be cut relative to the transepicondylar axis. Alternatively, there are other references that may also be used. Prepared bone surface 46 may have a medial-lateral slope, anterior-posterior slope or a compound slope that supports accurate leg movement and proper rotation of femoral component 48 over a range of motion.

Prosthetic component 50 may include one or more retaining features for coupling to prepared bone surface 46. In one example, prosthetic component 50, such as shown in FIG. 1, includes a keel 52, or stem, that is connected with base 56 and inserts into an opening 66 drilled into tibia 44. Opening 66 defines a location of prosthetic component 50 on prepared bone surface 46. Keel 52 positions, retains, and strengthens coupling of prosthetic component 50 to tibia 44. Keel 52 may be configured to be positioned within the medullary canal of tibia 44. Prosthetic component 50 may be rotated from a reference position. In one embodiment, a position of tibial prosthetic component 50 on tibia 44 is referenced to a medial third of the tibia tubercle. Rotation of prosthetic component 50 from this reference position may be measured and used as a reference for the inertial measurement unit 62. For example, rotation may be used to affect movement of the patella to prosthetic component 48. Rotation of prosthetic component 50 may also be used to affect alignment, balance, and contact point of prosthetic component 48 to prosthetic component 50. Once the position of tibial prosthetic component 50 is determined, bottom surface 56 of tibial prosthetic component 50 may be coupled to prepared bone surface 46 to further retain tibial prosthetic component 50 to tibia 44. Coupling of tibial prosthetic component 50 to tibia 44 typically comprises a mechanical attachment, an adhesive, or cement, or other coupling mechanism known in the art. In some examples, base 56 of prosthetic component 50 couples to prepared bone surface 46. In one embodiment, base 56 is approximately parallel to prepared bone surface 46.

In one example, inertial measurement unit 62 is or includes a MEMs (micro-electro mechanical) integrated circuit. For example, one or more of the gyroscopes or accelerometers may be or include a MEMs integrated circuit. The form factor of a MEMs gyroscope integrated circuit or MEMs accelerometer integrated circuit supports placement in a prosthetic component or coupling to a prosthetic component to measure alignment of the muscular-skeletal system. A MEMs integrated circuit is generally a solid state device formed using a photolithographic process. Such a MEMs integrated circuit may have a form factor that supports placement within a prosthetic component or a module that may be coupled to a bone surface.

In one example, a MEMs gyroscope has a resonating mass that shifts with angular velocity and outputs a signal corresponding to the angular velocity. For example, in some embodiments, the MEMs gyroscope may be configured to output a signal proportional to the angular velocity of the IMU 62. In one embodiment, a MEMs accelerometer has a mass-spring system that shifts in response to an exerted acceleration, e.g., counter to a bias of a spring in the mass-spring system. In one embodiment, a MEMs integrated circuit includes at least one gyroscope and at least one accelerometer and/or an accelerometer/gyroscope hybrid. MEMs integrated circuits may generally provide an analog or digital output.

In some examples, inertial measurement unit 62 may include or be connected with one or more load sensors. The one or more load sensors may be configured to record an amount of force applied to a portion of the implantable measurement device 50. Data from the one or more load sensors may be selectively processed by the implantable measurement device 50 to assist in determining the type of movement which the patient is performing, among other parameters.

In some examples, inertial measurement unit 62 may include or be connected with one or more temperature sensors. A temperature sensor may be configured to record the temperature of the implantable measurement device 50, the patient, or both. The temperature device 50 may be selectively activated, depending on the movement type the patient is performing.

In some examples, inertial measurement unit 62 may include or be connected with one or more magnetometer sensors. A magnetometer may measure a magnetic field strength and a magnetic field orientation. Data from the magnetometer may be processed by one or more processors on the implantable measurement device 50, or a remote system such as computer 43, for determining types of patient movement. The magnetometer may be selectively activated by the processor of the implantable measurement device 50 depending on the type of movement which the patient is performing.

In one example, measurement data from inertial measurement unit 62 is transmitted to a computer 43 configured to process and display movement metrics of the leg with implantable measurement device 50. Typically, inertial measurement unit 62 is connected with a mounting surface 63. The mounting surface 63 may correspond to a plane of two of the axes of inertial measurement unit 62. In one embodiment, the Xa-Ya plane of inertial measurement unit 62 is placed approximately parallel to prepared bone surface 46 of tibia 44 and corresponding parallel surfaces of prosthetic component 50. In one embodiment, inertial measurement unit 62 is placed in prosthetic component 50. The support surface 68 is used to support installation of the implantable measurement device 50. The implantable measurement device 50, with inertial measurement unit 62, may measure alignment of the leg, as well as the amount and types of activity a patient is performing. Implantable measurement device 50 may also include other sensors to provide measurement data on other parameters in proximity to the leg or to support installation of the implantable measurement device 50.

Inertial measurement unit 62 may also be placed in other prosthetic components or a module. The position of prosthetic component 50 may be aligned or referenced to a medial third of the tibia tubercle or any other suitable reference point. The orientation of the inertial measurement unit 62 may be placed such that the Z-axis Za is approximately normal to the upper surface of base 56. Implantable measurement device 50 incorporating inertial measurement unit 62, may locate the inertial measurement unit 62 such that the X-axis Xb aligned to an anterior-posterior direction of implantable measurement device 50 and having the Y-axis Yb aligned to the medial-lateral direction of implantable measurement device 50.

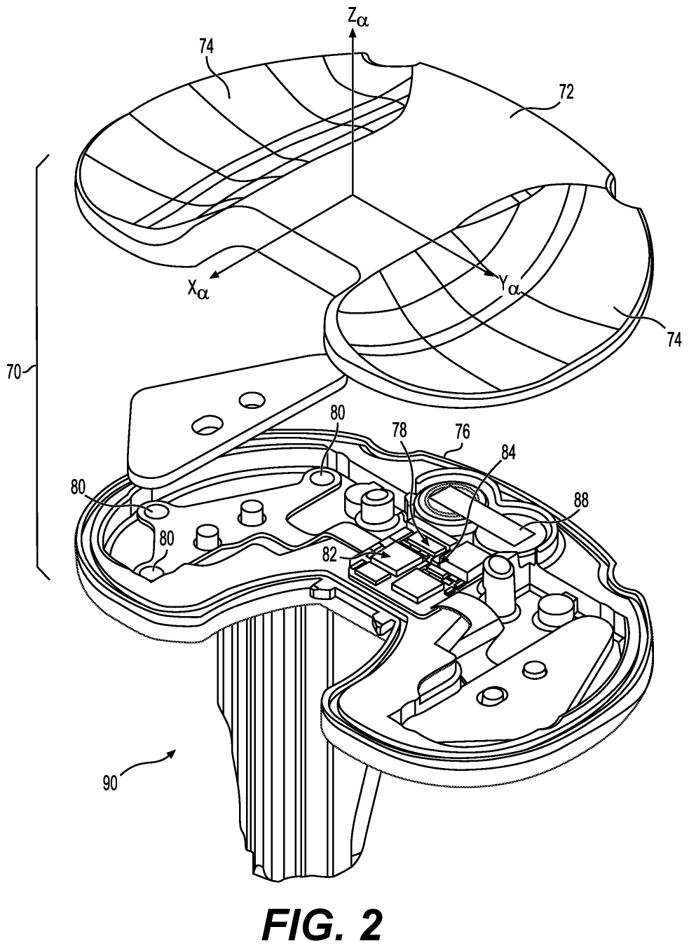

FIG. 2 is an illustration of an implantable measurement device 70 configured as a prosthetic component incorporating an inertial measurement unit 82 in accordance with an example embodiment. In general, inertial measurement unit 82 may be housed in the implantable measurement device 70 or coupled to the muscular-skeletal system to measure activity and/or alignment. Inertial measurement unit 82 may measure alignment of the muscular-skeletal system and/or measure bone position. Inertial measurement unit may measure the position and orientation of the joint (e.g., analyzing movement) to determine a type of activity that a patient may be performing. Inertial measurement unit 82 may have a predetermined orientation referenced to the implantable measurement device 70. The implantable measurement device 70 maybe a permanent prosthetic component configured as an implantable measurement device that monitors or measures alignment and/or activity of the joint long-term. For example, changes in alignment over time may indicate incorrect loading on the articular surfaces that long-term could accelerate wear if not corrected. In another example, the implantable measurement device 70 with the inertial measurement unit 82 may be configured to monitor activity, and, further, may distinguish between different movements and activities, such as between a target activity and a non-target activity. Although inertial measurement unit 82 is shown in a knee joint in this example, inertial measurement unit 82 is not limited to knee prosthetic components and may be applied similarly to other parts of the anatomy such as but not limited to the muscular-skeletal system, hip, shoulder, spine, ankle, elbow, wrist, fingers, toes, and wrist. Similarly, inertial measurement unit 82 may be placed in other knee components such as a patellar button, tibial prosthetic component, or femoral prosthetic component to support alignment measurement or other measurements.

In one embodiment, implantable measurement device 70 comprises a support structure 72 having articular surfaces 74, and a support structure 76. Support structures 72 and 76 couple together to form a housing. The housing includes at least one cavity for electronic circuitry and sensors. In one embodiment, a peripheral surface of support structure 72 couples to a peripheral surface of support structure 76. The surfaces may be coupled together via an adhesive that seals the cavity from the external environment. The interior and exterior of implantable measurement device 70 may be sterilized and stored in a package prior to use. Support structures 72 and 76 comprise a bio-compatible polymer such as polycarbonate, PEEK, or ultrahigh molecular weight polyethylene. The selected polymer may support loading applied by the muscular-skeletal system while providing a low friction surface for joint movement and reduced wear.

According to one or more embodiments, a measurement device comprises inertial measurement unit 82, sensors 80, electronic circuitry 78, a power source 88, e.g., housed in implantable measurement device 70. A remote system, such as computer 43, may be used with the implantable measurement device 70. For example, the remote system may be configured to display activity and/or measurement data provided by the sensors of inertial measurement unit 82. The remote system receives activity and/or measurement data from the sensors 80 and inertial measurement unit 82 via wired connection or wireless transmission. In some examples, wireless transmission may be short range, such as Bluetooth, and may be encrypted for security. In another example, wireless transmission may be performed via WiFi or a cellular network. The remote system may comprise a computer with a display to receive and process activity and/or measurement data from implantable measurement device 70. The computer may include software programs to support calculation and visualization of the measurement data. Alternatively, the remote system may be a microprocessor-based device capable of running software such as a smart phone or handheld device that allows a patient or other user to review measurement data transmitted from the implantable measurement device 70.

Implantable measurement device 70 may be a portion of a tibial prosthetic component 90. Tibial prosthetic component 90 may be configured to couple with a tibia. Articular surfaces 74 couple to condyles of a femoral prosthetic component and/or portions of a femur to support movement of the knee joint and the leg. Load sensors 80 maybe placed underlying articular surfaces 74. The implantable measurement device 70 may measure load and position of load applied to articular surfaces 74. The load applied to articular surfaces 74 is distributed through to the tibial prosthetic component 90. The surface area of the tibial prosthetic component 90 is greater in area than the condyle contact area of the femoral prosthetic component to articular surfaces 74.

As noted above, the implantable measurement device 70 includes inertial measurement unit 82 for measuring alignment of a leg relative to a mechanical axis of the leg, as well as record activity of a patient. The inertial measurement unit 82 comprises three gyroscopes and three accelerometers. A first gyroscope and a first accelerometer have a rotational axis aligned in an anterior-posterior direction of prosthetic component 70 corresponding to an axis Xa. A second gyroscope and a second accelerometer have a rotational axis aligned in a medial-lateral direction of in prosthetic component 70 corresponding to an axis Ya. A third gyroscope and a third accelerometer have a rotational axis perpendicular to an Xa-Ya plane corresponding to an axis Za.

Electronic circuitry 78 may be housed in the prosthetic component 70 with inertial measurement unit 82, and may be aligned as stated above regarding inertial measurement unit 82 or in another predetermined alignment. As mentioned above, inertial measurement unit 82 is a small form factor device that allows placement within the prosthetic component 70 or device that couples to the muscular-skeletal system. The sensors 80 may measure a parameter of the musculoskeletal system or measure a parameter in proximity to prosthetic component 70.

Electronic circuitry 78 is mounted on a printed circuit 84 that is centrally mounted in the cavity of prosthetic component 70. Electronic circuitry 78 is mounted in an area of prosthetic component 70 that has little or no joint loading, which may increase reliability of and may prevent damage to electronic circuitry 78. Load sensors 80 may be coupled to electronic circuitry 78 and underlie articular surfaces 74. In one embodiment, load sensors 80 and electronic circuitry 78 are coupled to a flexible and unitary printed circuit board (not shown). In one embodiment, load sensors 80 may be integrated into the printed circuit board (not shown) to simplify assembly, improve reliability, and increase performance of the implantable measurement device 70. Three or more load sensors 80 may be used measure a position where the load is applied on articular surfaces 74. In some examples, inertial measurement unit 82 may include an integrated temperature sensor. In other examples, the temperature sensor is separate from the inertial measurement unit 82 (not shown). In some examples, inertial measurement unit 82 may include a magnetometer sensor integrated with the inertial measurement unit 82. In some other examples, the magnetometer is separate from the inertial measurement unit 82 (not shown). The inertial measurement unit 82 may be mounted to printed circuit board 84 and couples to electronic circuitry 78. The inertial measurement unit 82 is mounted such that the three gyroscopes and three accelerometers are oriented in relation to the implantable measurement device 70.

Electronic circuitry 78 and inertial measurement unit 82 may be isolated from an external environment. Electronic circuitry 78 may include a power source 88, passive components, power regulation, power management circuitry, conversion circuitry, digital logic, analog circuitry, microprocessors, microcontrollers, digital signal processors, memory, ASICs, interface circuitry, or communication circuitry. In some examples, the electronic circuit 78 may be configured as a control system for the implantable measurement device 70.

According to one or more embodiments, techniques for determining an angle of the joint relative to a gravity vector via an accelerometer in combination with a gyroscope are performed using a 3D vector-based math approach. An anatomical reference frame of the tibia of a patient is determined relative to an inertial measurement unit 62, 82 of an implantable measurement device 50, 70, e.g., incorporating features of described above. Under such an approach, the measurement device 60 may have a predetermined reference frame for the inertial measurement unit 62, 82 with respect to the tibia anatomical reference frame, e.g., based on a calibration process associated with the implantable measurement device 50, 70.

Placement of the implantable measurement device 50, 70 may establish a center point and/or axis of the tibia relative to the inertial measurement unit 62, 82. According to one or more embodiments, the implantable measurement device 50, 70 may be rigidly placed in a position within the implantable measurement device 50, 70, e.g., in order to collect/generate calibration data for the implantable measurement device 50, 70 that may be taken into account when determining the angle of the implantable measurement device 50, 70. Calibration of the implantable measurement device 50, 70 may significantly increase accuracy of the angle determination.

Generally, error encountered in measurement from accelerometers and gyroscopes may include, for example, biases, scale-factor or scaling errors, and/or misalignment or skew errors. Bias error is typically associated with error present regardless of the forces or rates induced on the sensor, e.g., irrespective of the external input sensed by the accelerometer or gyroscope. Generally, bias error may represent the largest error source for an inertial measurement unit 62, 82, and may be the biggest contributor to the accuracy improvement when properly calibrated. Scaling errors are associated with how well an output of a sensor corresponds to an applied external action, e.g., force or rate input. Misalignment or skew errors may result from imperfect construction or alignment of the three sensing axes in the accelerometer or gyroscope.

In the various methods and operations below, various acts are described as performed or executed by a component discussed elsewhere in this disclosure, e.g., a component or device from FIG. 1 or 2. However, it should be understood that in various embodiments, various components discussed above may be used to perform acts including the acts discussed below. Further, it should be understood that in various embodiments, various steps may be added, omitted, and/or rearranged in any suitable manner.

In one example, a first calibration operation for the implantable measurement device 50 may include recording a plurality of measurements from the accelerometer and the gyroscope while moving the inertial measurement unit 62 between different positions and/or orientations. As used herein, the term “pose” generally encompasses a position and/or orientation, and may also include relative positions and/or orientations of various elements to each other, e.g., the position/orientation of the tibia 44 to the femur 42, of the inertial measurement unit 62 to the implantable measurement device 50, of the inertial measurement unit 62 to a vector of Earth's gravity, or the like. In an exemplary embodiment, the first calibration process may include moving the inertial measurement unit 62, e.g., when installed in the implantable measurement device 50, between fourteen different static poses: six poses so as to be oriented in the positive and negative direction for each of the Xb, Yb, and Zb, axes that define the reference frame of the implantable measurement device 50; and eight poses corresponding to diagonal vectors in four different Cartesian quadrants. However, it should be understood that, in various embodiments, any suitable number of poses in any suitable orientation may be used. In various embodiments, the inertial measurement unit 62 may be posed while installed in or outside of the implantable measurement device 50. The plurality of measurements taken during the posing of the inertial measurement unit 62 may be used to generate a respective skew, scaling, and bias matrix for each of the accelerometer and the gyroscope of the inertial measurement unit 62.

Determining the matrices above may include filtering the data collected from the accelerometer and/or gyroscope. For example, a Butterworth 2nd order filter may be applied. In some embodiments, a portion of the measurements, e.g., a predetermined number of measurements at a beginning or ending of a movement toward a pose, may be discarded.

The bias matrix for the gyroscope may be determined, e.g., by averaging raw data from static measurements included in the plurality of measurements associated with measurements collected at each pose. For example, the bias matrix may include, for each axis Xb, Yb, Zb, an offset based on an average of the static measurements for that axis from the plurality of measurements. The skew and scaling matrices for the gyroscope may be determined using optimization via a cost function. For example, a cost function may be associated with a comparison between an estimated gravity direction and a measured gravity direction determined using the accelerometer.

A second calibration operation may be performed to determine a reference frame of the inertial measurement unit 62 relative to, for example, a bottom surface 56 of a housing 50 of the implantable measurement device 50. As noted above, the margin for a tibial varus/valgus angle is generally one to three degrees. As such, even a minor offset due to materials or machining tolerances affecting the orientation of the inertial measurement unit 62 when housed in the measurement device may be significant. By accounting for any relative orientation between a reference frame of the inertial measurement unit 62 and the implantable measurement device 50 via the second calibration operation, such error may be accounted for and/or reduced.

In some embodiments, a second calibration operation, e.g., a registration operation, may be performed. For example, a reference frame of the inertial measurement unit 62 may, via the second calibration operation, be registered in two dimensions. The second calibration operation may include, for example, collecting a plurality of measurements from the accelerometer and the gyroscope of the inertial measurement unit 62 at different static poses. In an exemplary embodiment, eight static poses of the implantable measurement device 50 with the inertial measurement unit 62 installed therein are used, the poses being forty-five degrees apart in rotation about the z-axis. The data from the gyroscope may be used to detect when the inertial measurement unit 62 is moving, e.g., to identify a portion of time when the inertial measurement unit 62 is static corresponding to each pose. At each pose identified based on a respective portion of time when the inertial measurement unit 62 is static, a gravity vector may be determined based on the data from the accelerometer during the corresponding portion of time. The points defined by the gravity vectors may then be fitted to a circle in three-dimensional space, and a center, normal vector and radius for the circle may be determined, which may then be used to determine an average gravity direction across the various poses, which, for example, may be used to define the reference frame of the inertial measurement unit 62 (Xa, Ya, and Za) relative to the reference frame of the implantable measurement device 50. In some embodiments, the second calibration operation, such as of the kind in the foregoing example, may at least partially correct for misalignment between a rotation axis of the inertial measurement unit 62 and a direction of gravity. It should be understood that the foregoing calibration/registration examples are illustrative only, and that any suitable technique for reducing misalignment via calibration or the like may be used.

In some embodiments, the calibration data, e.g., the matrices and reference frame data discussed above, may be stored on a memory onboard the implantable measurement device 70. In some embodiments, such data may be stored in a database in an entry associated with the implantable measurement device 70 and/or inertial measurement unit 62.

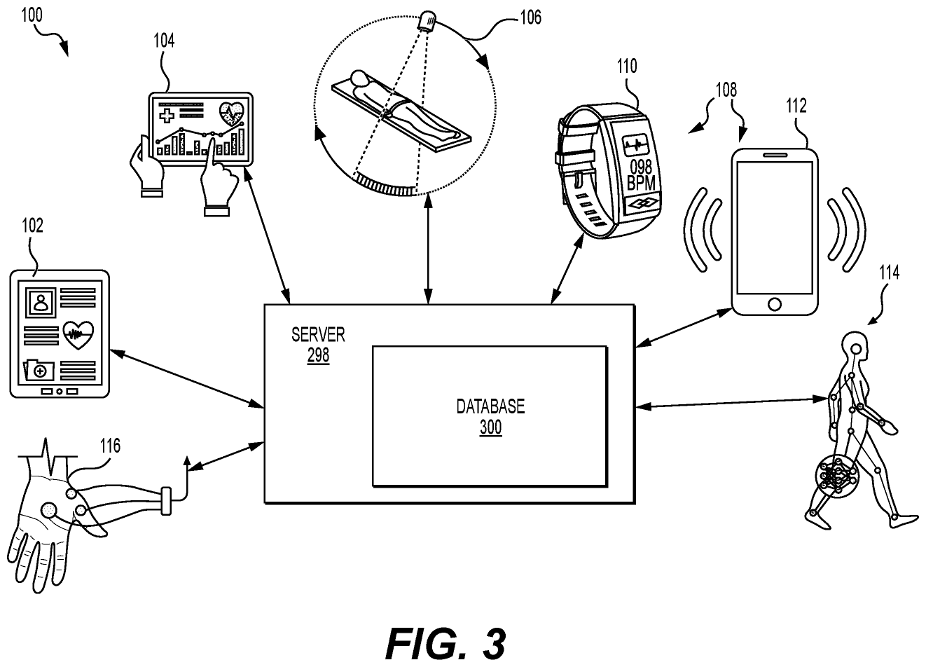

Referring to FIG. 3, the implantable measurement device may be in communication with one or more devices configured to send and receive data between database 300 of network system 100. The network system 100 may include electronic devices storing electronic medical records (EMR) 102, patient/user interfaces or applications 104 such as tablets, computers, and cellular phones 112, diagnostic imaging systems 106, mobile devices 108, a motion sensor, pressure sensor, and/or kinesthetic sensing systems 114, and electromyography or EMG systems 116. The devices of the network system 100 may each include one or more communication modules (e.g., WiFi modules, Bluetooth modules, etc.) configured to transmit patient data to and from the implantable measurement device and the database 300, to each other, or a combination thereof. In some examples, database 300 is connected with server 298.

The network system 100 may collect patient reported data, practitioner assessments, etc. using EMR 102. For example, EMR 102 may be used to collect data on demographics, medical history, biometrics, and information about procedures. Patient and/or user interfaces may be implemented on mobile applications and/or patient management websites or interfaces such as OrthologIQ®. Patient interfaces may present questionnaires, surveys, or other prompts for patients to enter psychosocial information such as perceived or evaluated pain, stress level, anxiety level, feelings, and other patient reported outcome measures (PROMS). Practitioners may also report psychosocial information (e.g., qualitative assessments or evaluations) via patient interfaces or other interfaces. Patients may also report lifestyle information via patient interfaces. These patient interfaces may be executed on other devices disclosed herein (e.g., using mobile devices 108 or other computers).

Network system 100 may collect imaging information from diagnostic imaging systems 106, which may include computed tomography (CT) scans, magnetic resonance imaging (MRI), x-rays, radiography, ultrasound, thermography, tactile imaging, elastography, nuclear medicine functional imaging, positron emission tomography (PET), single-photon emission computer tomography (SPECT), etc. The system 20 may use these diagnostic imaging systems 106 to collect bone imaging information, including morphology and/or anthropometrics fractures, and bone density (e.g., bone mineral density or bone marrow density).

Mobile devices 108 may include smartwatches 110, smartphones 112, tablets, and other devices known in the art. Mobile devices 108 may execute patient interfaces. In some examples, mobile devices 108 may include sensors and/or applications, which the system 20 may use to collect biometrics and other types of patient specific data. For example, mobile devices 108 (e.g., FitBit, Apple Watch, Hexoskin, Polaris strap, iPhone, etc.) may use cameras, light sensors, barometers, global positioning systems (GPS), accelerometers, temperature sensors (e.g., battery temperature sensors), and/or pressure sensors. In some examples, mobile devices 108 may measure heart rate, electrocardiogram data, breathing rate, temperature, oxygenation, sleep patterns, and also activity frequency and intensity.

Network system 100 may use EMG systems 116 to collect EMG data. EMG systems 116 may include one or more electrode attached to skin or muscle to measure electrical activity and/or responses to nerve stimulation. The system 100 may use EMG data to determine nerve damage or disease information. EMG data may include information on muscle activity of various body segments including knee, hip, ankle, tibialis anterior, foot, lower back, shoulder, wrist, elbow, forearm, neck, etc.

The Network system 100 may use motion sensor and/or kinesthetic sensing systems 114, which may include motion capture (mocap) systems, external motion sensors, wearable sensors, and/or sensors machine vision (MV) technology. Motion sensor systems 114 may measure motion using an optical or light sensor, an accelerometer, a gyroscope, a magnetometer, a compass, barometer, a global positioning system (GPS), a pressure sensor, the like, or a combination thereof.

The system 100 may use motion capture systems, which may include markerless motion capture systems and other motion sensors (e.g., wearable sensors) to collect kinematics and range of motion data. External motion sensors may include cameras, optical sensors, infrared sensors, ultrasonic sensors, etc. mounted, for example, in an operating room to monitor motion, heat, etc.

Wearable sensors 114 may include heart-rate monitors, some mobile devices 108 (e.g., smartwatch 110), and other sensor systems configured to be worn by a patient and track movement (e.g., travel movement and kinematics of anatomy, such as joint motion). Wearable sensors 114 may include accelerometers, GPS chips, acoustical ranging, magnetometer, inclinometers, hybrid sensors, MEMs devices, the like, or a combination thereof. Wearable sensors 114 may also include MotionSense sensors, ZipLine sensors, and/or pedometers. Wearable sensors 114 may monitor more than motion, such as pressure, temperature, sweat/perspiration, input related to stress, input related to air circulation, air purity or quality of an environment, etc. Wearable sensors 114 may include pressure insole sensors and/or sensored shoes configured to measure pressure, a pressure distribution, a center of pressure, etc. when a user steps. Such wearable sensors 114 may also measure acceleration or force as a user lifts a leg to take a step. Pressure data from pressure insole sensors or sensored shoes may be used to determine or evaluate balance, heel strike, and/or push-off forces, which may be used to determine or evaluate frailty, fall risk, compensatory gait, and overall function.

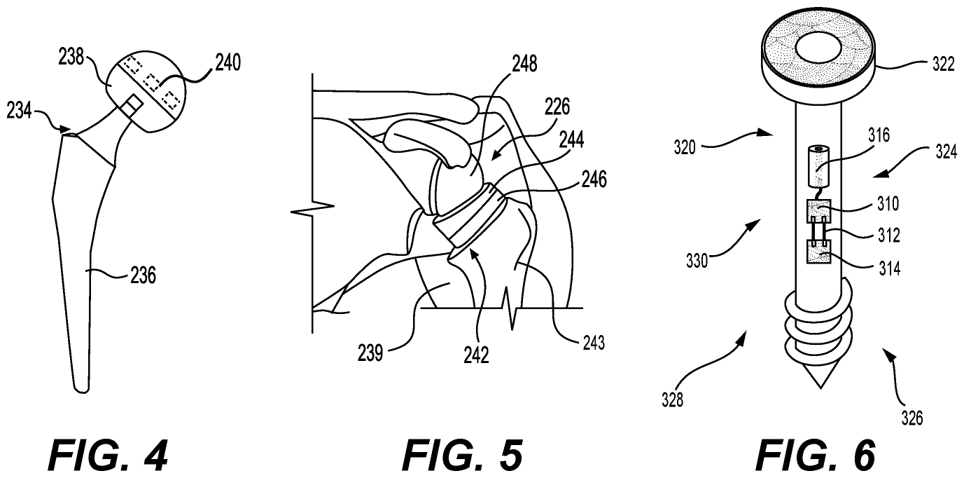

Turning to FIGS. 4-6, different implantable measurement devices may be incorporated into different regions of a patient. For example, FIG. 4 and FIG. 5 each illustrate an example ball-and-socket surgical implant configured as an implantable measurement device. Similarly, FIG. 6 illustrates one example of a screw, which may be used as a spinal implant, configured as an implantable measurement device.

FIG. 4 illustrates on example of a femoral prosthetic device 234 that may include (e.g., within the ball joint 238 and/or the stem 236) one or more sensors 240 such as strain gauge sensors, IMUs, optical sensors, pressure sensors, load cells, ultrasonic sensors, acoustic sensors, and/or sensors configured to sense synovial fluid and/or detect an infection (e.g., via blood glucose, body temperature, sleep disturbances, heart rate variability, etc.). The femoral prosthetic device 234 may be configured to measure, via the one or more sensors 240, magnitude, location, and/or direction of forces placed on the femoral prosthetic device 234 (e.g., on ball joint 238) and/or a position, orientation, speed, acceleration, etc. of the femoral prosthetic device 234 (e.g., ball joint 238).

For example, the one or more sensors 240 may include three strain gauge sensors positioned circumferentially around a central circuit board of the ball joint 238 and positioned at an equal distance from a center of the ball joint 238 and/or some other reference. Each strain gauge sensor may be spaced equally from each adjacent sensor. Different strains, loads, pressures, forces, etc. measured by each strain gauge sensor may be processed to determine a load magnitude and location of the load applied to the ball joint 238. Alternatively or in addition thereto, the femoral stem 236 may include sensors to measure, for example, rotation of the femur about the hip joint and/or ball joint 238 and/or whether the femoral stem 236 is moving (e.g. loosely coupled to the femur, etc.). The measured strains and/or other data may be transmitted to the system 20 or another computing platform to calculate load parameters, such as magnitude, location, direction, etc. of an applied load, force, etc. of a joint (e.g., hip joint) in real time, which may then be visualized on a display.

FIG. 5 illustrates one example of a shoulder prosthetic system 226 that may include a glenoid prosthetic component or glenoid sphere 248, a humeral prosthetic component 242, and a measurement device or insert 244. The glenoid sphere 248 may be configured to be coupled to a prepared bone surface of a scapula 239 of the scapula 239. The glenoid sphere 248 may have an anchor or stem to support an attachment (e.g., via screws) to scapula 239. The glenoid sphere 248 has an external, convex curved surface configure to couple to the measurement device 244. The humeral prosthetic component 242 may be configured to couple to a prepared bone surface of a humerus 243. The humeral prosthetic component 242 may have a humeral tray 246 configured to couple with the measurement device 244. The measurement device 244 may have an external, concave curved surface configured to couple to the external, convex curved surface of the glenoid sphere 248.

The glenoid sphere 248, humeral prosthetic component 242, and/or measurement device 244 may include at least one sensor such as strain gauge sensors, IMUs, optical sensors, pressure sensors, load cells, ultrasonic sensors, acoustic sensors, and/or sensors configured to sense synovial fluid and/or detect an infection (e.g., via blood glucose, body temperature, sleep disturbances, heart rate variability, etc.). The one or more sensors may be configured to measure, via the one or more sensors, magnitude, location, and direction of forces placed on the glenoid sphere 248, humeral prosthetic component 242, and/or measurement device 244 and/or a position, orientation, speed, acceleration, etc. of the glenoid sphere 248, humeral prosthetic component 242, and/or measurement device 244.

FIG. 6 illustrates one example of an implant screw system 320. Implant screw system 320 may comprise a screw 328 including a measurement system 330. Screw 328 may comprise a head 322, a cannulated shaft 324, and threads 126 for insertion into a bone or other tissue during a surgery. Measurement system 330 may comprise an integrated device 316 and printed circuit boards 310 and/or 314 connected by circuitry 312. Electronic circuitry and sensors (e.g., accelerometer, gyroscope, load, temperature, magnetometer, etc.) may be mounted on printed circuit boards 310, 314, and coupled together to form measurement system 330. Circuit boards 310 and 314 may be flexible for ease of installation into the cavity of cannulated shaft 124. In the example, the walls of cannulated shaft 324 are transparent to show measurement system 330 housed within cannulated shaft 324. Alternatively, circuit boards 310 and 314 and the electronic circuitry may be rolled up into a cylinder to fit within cannulated shaft 324. Any number of circuit boards 310, 314 may be incorporated into measurement system 330.

Integrated device 316 may be a battery or other power source to power the electronic circuitry of implant screw system 320. The measurement process may comprise generating quantitative measurement data and/or providing therapy in proximity to implant screw system 320.

### Sensor Control

The implantable measurement device is selectively controlled to adjust data collection, processing, and power consumption of the plurality of sensors described above. In some examples, the implantable measurement device collects movement data from a first sensor and analyzes the movement data to determine the type of movement that the patient is performing. In some examples, the implantable measurement device may be configured to distinguish between target and non-target patient activities, explained further below. When the implantable measurement device determines that the patient is performing the target activity, based on data from the first sensor, the implantable measurement device may turn on one or more additional sensors to collect additional movement or other data. When the implantable measurement device determines that the patient movement is performing non-target activities, only the first sensors are powered.

Further, in some examples, one sensor (e.g., accelerometer) may continuously collect data in order to filter and smooth data for more accurate identification of target activities. By continuously collecting data, the one or more processors of the implantable measurement device may “reduce noise” in the data to more easily identify when a patient is performing a target activity. In other examples, data filtering and smoothing may be selectively activated and deactivated, based on the battery level, whether the patient is performing the target activity, or both. Additionally, the sensor sampling rate and/or processing may be adjusted, which may prolong battery life, described further below. For example, the sensor sampling rate may be increased or decreased. In another example, the implantable measurement device may be configured to adjust the amount of processing of the first sensor, second sensor, or both by activating and deactivating firmware associated with processing the data of the sensors.

To determine whether the target activity is being performed, the implantable measurement device may analyze sensor data and compared the data with one or more threshold sensor values. A threshold sensor value may be a value associated with one or more sensors, which, when exceeded indicates that the target activity may be being performed.

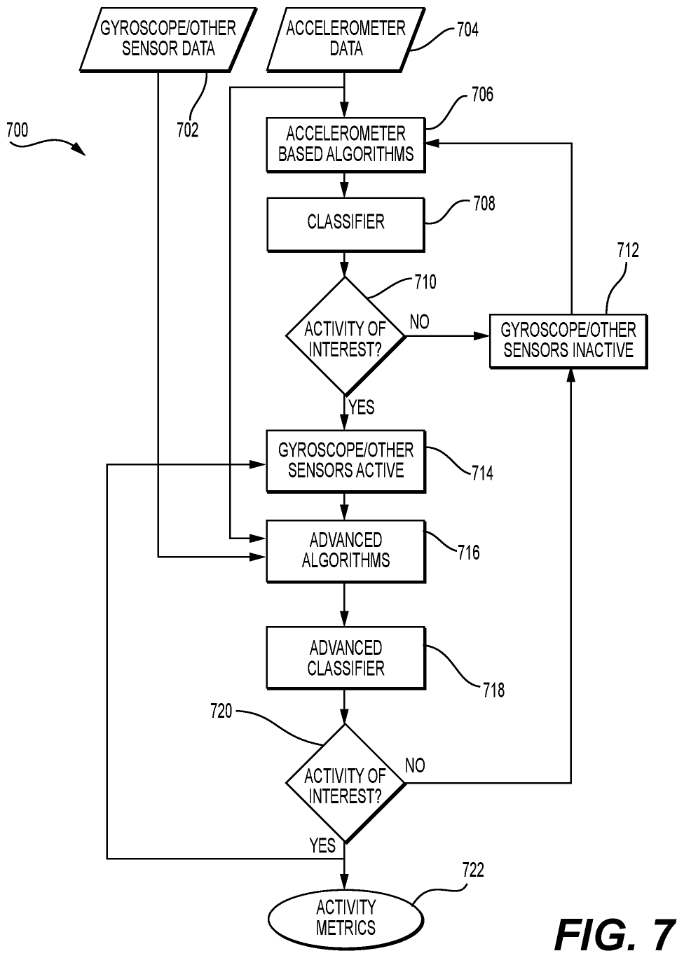

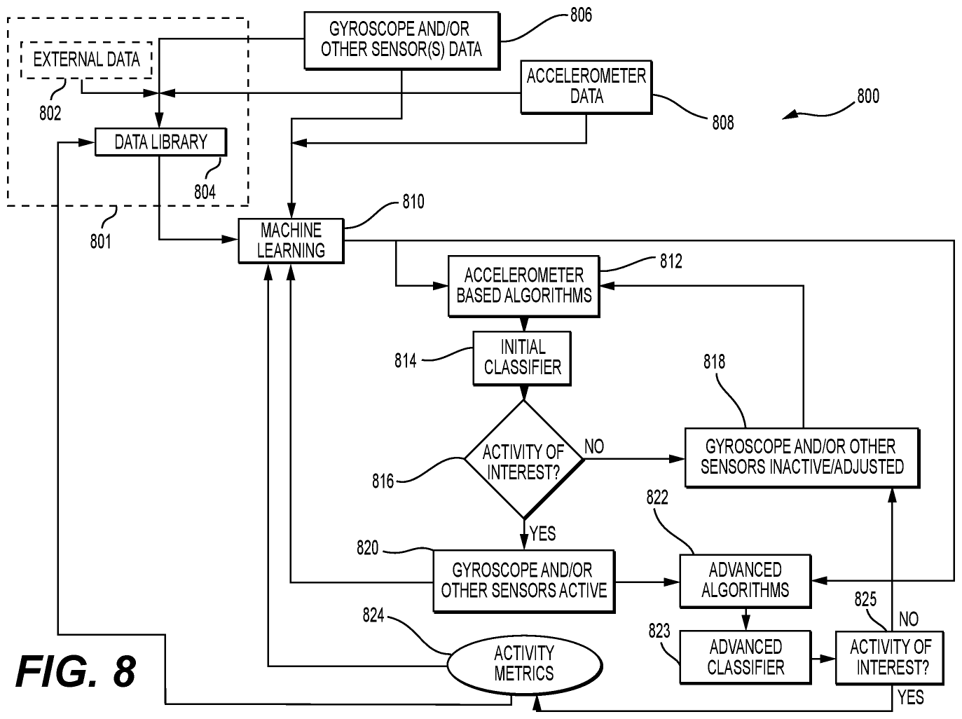

By selectively controlling the sensors of the implantable measurement device, power consumption and battery power may be conserved. In some examples, one or more sensors, one or more processors, one or more sampling rates, or a combination thereof may be controlled in order to conserve battery power, processing power, or both. FIGS. 7 and 8 each illustrate one example of a control method to selectively control the sensors of the implantable measurement device.

FIG. 7 illustrates one example of a control method 700 of an implantable measurement device. Control method 700 of the implantable measurement device may be performed partially or completely on an implantable measurement device (described above). In this example, the implantable measurement device includes at least one first sensor (e.g., accelerometer) and at least one second sensor (e.g., gyroscope). In other examples, a plurality of first sensors, such as accelerometers may be included. Similarly, in some examples, a plurality of second sensors may be used. Some examples of second sensors may be a gyroscope, a magnetometer, a temperature sensor, a load sensor, the like, or a combination thereof. Additionally, other sensors one of ordinary skill in the art would appreciate are also contemplated. In the example of FIG. 7, the first sensor may be an accelerometer, and the second sensor may be a gyroscope. The implantable measurement device collects accelerometer data 704 as a patient moves.

Accelerometer data 704 may be data associated with one or more accelerometers on the implantable measuring device. In some examples, there may be only one accelerometer, which would only output data associated with one axis. As described above, in some examples, there may be one accelerometer placed on each of the X-axis, Y-axis, and Z-axis such that a patient movement of the implantable measure device in any of the X-axis, Y-axis, or Z-axis would result in recording the acceleration along each axis.

The accelerometer data 704 may output data values associated with movement types. In some examples, the output data values may correspond with a speed of movement in one of the X-axis, Y-axis, and Z-axis. For example, when the implantable measurement device is located in a hip, a knee, and/or an ankle, an acceleration in the X-axis direction for a sample period (e.g., 3 seconds; 5 seconds), may indicate that the patient is jogging or running. Similarly, in some examples, when an acceleration in the Y-axis direction and/or Z-axis direction is output for a sample period (e.g., 3 seconds; 5 seconds), the patient may be jogging, running, riding a bike, ascending stairs, descending stairs, and/or some other aerobic activity. In another example, when the implantable measurement device is located in a shoulder, when acceleration in the Y-axis and/or Z-axis is output for a sample period, the patient may be extending their arm outward or overhead. In some examples, the implantable measurement device may be configured to track movement of the patient and determine an angle which the implantable measurement device has moved relative to a vector of Earth's gravity.

Gyroscope data/other sensor data 702 may be data associated with a gyroscope and/or other sensors, such as a load sensor, a temperature sensor, a magnetometer, or a combination thereof. In some examples, the implantable measurement device may only use a gyroscope (described above). In other examples, a gyroscope and one or more additional sensors may be used to collect data.

Gyroscope data/other sensor data 702 may be used to determine additional movement characteristics and/or confirm that a particular movement (e.g., target activity) is being performed. In some examples, such as when the gyroscope data/other sensor data 702 is gyroscope data, the implantable measurement device may be configured to determine an angle of the implantable measurement device relative to a vector of Earth's gravity. In some examples, such as when the implantable measurement device is located with a knee or a hip, when an angle of the implantable measurement device exceeds a certain angle during a sample time period, the patient may be performing a target activity. In some examples, the may angle be between 0 degrees and 120 degrees held over the sample time period. More particularly, the angle may be 10 degrees, 15 degrees, 30 degrees, 45 degrees, 65 degrees or even 90 degrees or more during the sample time period. Further, the implantable measurement device may be configured to distinguish between target activities based on the angle of the implantable measurement device associated with the knee joint and/or hip joint. In one example, a pitch angle of greater than 75 degrees of an implant in the hip or the knee during one or more sample time periods may indicate that the patient is in a prone or supine position. In another example, a pitch angle of greater than 75 degrees of an implant in the hip or the knee during one or more sample time periods, along with a trajectory of the implant may indicate that the patient is performing a long arc quad exercise. In another example, a knee implant crossing a 20 degree and a 60 degree flexion multiple times during a sample time period of 2 seconds to 4 seconds may indicate ambulation. In some examples, when the implantable measurement device is configured as a shoulder or spine implant, when an angle of the implantable measurement device exceeds a certain angle, the patient may be performing a target activity. In one example, when the implantable measurement device is configured as a spine implant, and the angle of the implant exceeds 25 degrees over the sample time period, the patient may be bending over, which may be considered a target activity. The determined angle may be different depending on the location of the spine implant in the patient spine. In another example, when the implantable measurement device is configured as a shoulder implant, and the angle of the implant exceeds 30 degrees over the sample time period, the patient may be reaching out or over head, which may be considered a target activity. Although some examples described above list particular angles of movement, any angle is contemplated as a threshold angle to determine a target activity. Further, the threshold angle may be adjusted or changed based on each patient and their specific characteristics. In some examples, a patient's height may impact threshold values (e.g., acceleration), since longer legs or a higher BMI might create a larger acceleration for similar activities. In another example, a patient with long legs may have lower hip angles since each stride covers a larger distance than a patient with shorter legs. Longer leg length may also affect angular velocities and step frequencies since step length is increased, resulting in less steps. The impact of acceleration in the z-direction (vertically) will be lower for shorter patients than taller patients (e.g., toddler compared with an adult). Similarly, differences may be found in patients with a different body habitus. In some examples, the thresholds may also change with recovery time, such as time post-op, gait performance, knee range of motion (e.g., collected via other sensors), a combination thereof, or the like.

Accelerometer data 704 may be processed by initial analysis 706, which may be configured to be programmed to determine whether the activity of interest 710 is being performed by the patient. In some examples, an initial analysis of accelerometer data 704 is performed by accelerometer-based algorithms 706. Accelerometer-based algorithms may be stored on the implantable measurement device and executed by one or more processors. In some examples, the accelerometer-based algorithms 706 may be a manually created heuristic algorithm that analyze accelerometer data 704 to determine an output value. Put differently, the accelerometer-based algorithms 706 may include a set of rules to determine an output value based on the accelerometer data 704, which will be analyzed against a threshold value for identifying whether a patient movement is a target activity. In some examples, the implantable measurement device, generating buffer data, may collect accelerometer data 704 continuously. The buffer data may be used to filter and reduce “noise” by collecting and averaging the data to improve the accuracy of identifying patient movements. In some examples, accelerometer data 704 may be collected from a single accelerometer. In other examples, accelerometer data 704 may be collected from a plurality of accelerometers. In some examples, there may be one accelerometer positioned on the x-axis, y-axis, and z-axis, respectively.

To determine the activity of interest 710 (also known as target activity), the accelerometer data 704 is processed by accelerometer-based algorithms 706 and the output value is compared with a threshold value at initial classifier step 708. At initial classifier step 708, the output value of the accelerometer-based algorithm is compared with a threshold value corresponding to an activity of interest. The threshold value may be a value assigned with a specific type of movement.

In one example, after accelerometer data 704 is processed by the implantable measurement device with the accelerometer-based algorithms 706, the output value may be below the threshold value of initial classifier step 708, indicating that the patient movement is not the target activity. When the output value is less than the threshold value, classifier step 708 determines that the patient movement is not the target activity, and keeps the gyroscope and/or other sensors inactive. The accelerometer may continue to record patient movement, as well as, continually monitor and analyze the data to determine whether the patient is transitions their movement to the target activity.

In another example, after the accelerometer data 704 is processed by the implantable measurement device with the accelerometer-based algorithms 706, the output value may be above the threshold value of classifier step 508, indicating that the patient movement is the target activity. After determining that the patient movement is performing the target activity, the implantable measurement device may turn on the at least one other sensor (e.g., gyroscope) at step 714.

When the initial classifier 708 has identified that accelerometer data 704 corresponds to the target activity at step 710, the gyroscope may be turned on at step 714 to collect gyroscope data 702. Gyroscope data 702 is processed by advanced algorithms 716. Advanced algorithms may utilize both accelerometer data 704 and gyroscope data 714 to determine whether the activity of interest is being performed, and to collect additional patient data.

Accelerometer data 704 and gyroscope data 714 may be analyzed by advance algorithms 716 to determine whether the patient is performing the target activity. Similar to initial classifier step 708, advanced classifier step 718 compares an output value of advance algorithms 716 with an advanced threshold value. In some examples, the advanced threshold value may be a single value. In other examples, the advance threshold value may be multiple values associated with each type of data collected by the implantable measurement device (e.g., gyroscope data 702, accelerometer data 704). In one example, a first advanced threshold value may correspond to accelerometer data 704, and a second advanced threshold value may correspond to gyroscope data 702. In some examples, the output value of the advanced algorithms corresponding to the accelerometer data 704 and the output value of the advanced algorithms corresponding to the gyroscope data 502 may both be required to be above the first advanced threshold value and the second advanced threshold value, respectively, in order to signal that activity of interest 520 (e.g., target activity) is being performed by the patient. In another example, only one of the accelerometer data 704 or the gyroscope data 702 may need to be above their respective advanced threshold values at advanced classier step 718.

When the implantable measurement device determines that the patient is no longer performing the activity of interest 720, the gyroscope and/or other additional sensors are deactivated, and the implantable measurement device continues to collect accelerometer data 704. As shown in the diagram of FIG. 7, when the implantable measurement device determines that the activity of interest 720 is not being performed, the control method 700 continues to run steps 704 to 710 until the accelerometer data 704 signals that an activity of interest 510 is being performed by the patient. By keeping the gyroscope and/or other sensors deactivated while non-target activities are being performed, power and processing power is saved by the implantable measurement device.

When the implantable measurement device determines that the patient is performing the activity of interest 720, the gyroscope and/or other additional sensors are continued to be powered, and accelerometer data 704 and gyroscope data 702 are collected and used to record activity metrics 722. Activity metrics 722 may be collected regarding the target activity. Activity metrics 722 may be recorded and displayed on a remote device wirelessly connected with the implantable measurement device.

In some examples, the accelerometer data 704 may output data values associated with movement types, which is then compared with a determined threshold value. The threshold value may be an angle, an acceleration, a force, the like or a combination thereof. In some examples, the output data values may correspond with an acceleration of movement in one of the X-axis, Y-axis, and Z-axis. For example, when the implantable measurement device is located in a hip, a knee, and/or an ankle, an acceleration in the X-axis direction for a sample time period (e.g., 3 seconds; 5 seconds), may indicate that the patient is jogging or running. Similarly, in some examples, when an acceleration in the Y-axis direction and/or Z-axis direction is output for a sample time period (e.g., 3 seconds; 5 seconds), the patient may be jogging, running, riding a bike, ascending stairs, descending stairs, and/or some other aerobic activity. In another example, when the implantable measurement device is located in a shoulder, when acceleration in the Y-axis and/or Z-axis is output for a sample time period, the patient may be extending their arm outward or overhead. In some examples, the implantable measurement device may be configured to track movement of the patient and determine an angle which the implantable measurement device has moved relative to a vector of Earth's gravity. In other examples, the implantable measurement device is configured to track the direction of movement, which may be analyzed to determine what kind of movement the patient is performing.

In some examples, gyroscope data/other sensor data 702 may be used to determine additional movement characteristics and/or confirm that a particular movement (e.g., target activity) is being performed and used to set a threshold value. In some examples, such as when the gyroscope data/other sensor data 702 is gyroscope data, the implantable measurement device may be configured to determine an angle of the implantable measurement device relative to a vector of Earth's gravity. In some examples, such as when the implantable measurement device is located with a knee, a hip, or ankle, when an angle of the implantable measurement device exceeds a certain angle (e.g., threshold angle), the patient may be performing a target activity. In some examples, the may be between 0 degrees and 120 degrees. In some examples, such as when the implantable measurement device is located with a knee or a hip, when an angle of the implantable measurement device exceeds a certain angle during a sample time period, the patient may be performing a target activity. In some examples, the may angle be between 0 degrees and 120 degrees held over the sample time period. More particularly, the angle may be 10 degrees, 15 degrees, 30 degrees, 45 degrees, 65 degrees or even 90 degrees or more during the sample time period. Further, the implantable measurement device may be configured to distinguish between target activities based on the angle of the implantable measurement device associated with the knee joint and/or hip joint. In one example, a pitch angle of greater than 75 degrees of an implant in the hip or the knee during one or more sample time periods may indicate that the patient is in a prone or supine position. In another example, a pitch angle of greater than 75 degrees of an implant in the hip or the knee during one or more sample time periods, along with a trajectory of the implant may indicate that the patient is performing a long arc quad exercise. In another example, a knee implant crossing a 20 degree and a 60 degree flexion multiple times during a sample time period of 2 seconds to 4 seconds may indicate ambulation. In some examples, when the implantable measurement device is configured as a shoulder or spine implant, when an angle of the implantable measurement device exceeds a certain angle, the patient may be performing a target activity. In one example, when the implantable measurement device is configured as a spine implant, and the angle of the implant exceeds 25 degrees over the sample time period, the patient may be bending over, which may be considered a target activity. The determined angle may be different depending on the location of the spine implant in the patient spine. In another example, when the implantable measurement device is configured as a shoulder implant, and the angle of the implant exceeds 30 degrees over the sample time period, the patient may be reaching out or over head, which may be considered a target activity. Although some examples described above list particular angles of movement, any angle is contemplated as a threshold angle to determine a target activity. Further, the threshold angle may be adjusted or changed based on each patient and their specific characteristics. In some examples, a patient's height may impact threshold values (e.g., acceleration), since longer legs or a higher BMI might create a larger acceleration for similar activities. In another example, a patient with long legs may have lower hip angles since each stride covers a larger distance than a patient with shorter legs. Longer leg length may also affect angular velocities and step frequencies since step length is increased, resulting in less steps. The impact of acceleration in the z-direction (vertically) will be lower for shorter patients than taller patients (e.g., toddler compared with an adult). Similarly, differences may be found in patients with a different body habitus. In some examples, the thresholds may also change with recovery time, such as time post-op, gait performance, knee range of motion (e.g., collected via other sensors), a combination thereof, or the like.

FIG. 8 illustrates another example operation of an implantable measurement device. Control method 800 of an implantable measurement device may be performed in-part or wholly on the implantable measurement device. In the example illustrated in FIG. 8, a portion of the control method 800 may take place external to the implantable measurement device, such as indicated at external processing 801. External processing 801 may include external data 802. External data 802 may include a library of patient data from multiple patients (data library 804). In some examples, the data library 804 is gathered and cataloged by performing specific movements and activities with motion capture techniques and data acquisition corresponding to a suite of sensors (e.g., accelerometer, gyroscope, load sensor, temperature sensor, magnetometer, etc.). Collecting the movement data from a plurality of patients may be supervised and guided by movement professionals to ensure the data input into the data library 804 is directed to the specific target movements. The movement data saved to the data library 804 includes data tags of specific sensor data corresponding to particular movements of the target activity. Data library 804 may include one or more prior patient datasets. Additionally, in some examples, the prior patient datasets may include cataloged patient features associated with specific movements and activities, such as age, height, weight, knee size, bone size, stride length, gait, type of implant, the like, or any other patient features that would be appreciated by one of ordinary skill in the art. External data 802, as well as sensor data from the implantable measurement device may be sent to data library 804 for analysis and classification that may use machine learning algorithm 810 to further refine activity classification of the patient movement, explained further below.

Similar to FIG. 7, control method 800 of FIG. 8 collects and analyzes at least a first sensor data and a second sensor data. In some examples, one or more of the sensors may be powered off as a default setting. In another example, one of the one or more sensors may be continuously powered. In this example, the powered sensor may be used to signal when one or more additional sensors may be powered on. In another example, processing associated with one or more sensors may be increased or decreased, based on patient activity, battery power, or both. For example, a first sensor is an accelerometer, and the power level of the implantable measurement device is low, the accelerometer may take less samples of the patient movement than when the power level of the implantable measurement device is high. In another example, a first sensor may be an accelerometer, and the implantable measurement device determines that the patient movement corresponds to a target activity based on the accelerometer data and, when the movement corresponds to the target activity, turns on one or more additional sensors, such as a gyroscope, a load sensor, a magnetometer, a temperature sensor, or a combination thereof to record data associated with the target activity.

In one non-limiting example, such as shown in FIG. 8, a first sensor data may be accelerometer data 808, and a second sensor data may be gyroscope data 806. Other types of sensors are contemplated such as load, temperature, magnetic, or any other type of sensor one of ordinary skill in the art would use for recording kinematic movements of a patient. When the first sensor and/or second sensor collect data, the sensor data is input into a machine learning algorithm 810. Machine learning algorithm 810 may be loaded onto the processor of the implantable measurement device. In another example, one or more external devices in communication with the implantable measurement device that may include the machine learning algorithm 810. As shown in FIG. 8, accelerometer data 808 and gyroscope and/or other sensor data 806 is processed by machine learning algorithm 810.

Accelerometer data 808 may be data associated with one or more accelerometers on the implantable measuring device. In some examples, there may be only one accelerometer, which would only output data associated with one axis. As described above, in some examples, there may be one accelerometer placed on each of the x-axis, y-axis, and z-axis such that a patient movement of the implantable measure device in any of the x-, y-, or z-axes would result in recording the acceleration along each axis.

In some examples, the accelerometer data 808 may output data values associated with movement types, which is then compared with a determined threshold value. The threshold value may be an angle, an acceleration, a force, the like or a combination thereof. In some examples, the output data values may correspond with an acceleration of movement in one of the X-axis, Y-axis, and Z-axis. For example, when the implantable measurement device is located in a hip, a knee, and/or an ankle, an acceleration in the X-axis direction during the sample time period, may indicate that the patient is jogging or running. Similarly, in some examples, when an acceleration in the Y-axis direction and/or Z-axis direction is output during the sample time period, the patient may be jogging, running, riding a bike, ascending stairs, descending stairs, and/or some other aerobic activity. In another example, when the implantable measurement device is located in a shoulder, when acceleration in the Y-axis and/or Z-axis exceeds a determined angle or other value over the sample time period, the patient may be extending their arm outward or overhead. It is contemplated that the threshold value may be set and/or changed based on the patient. In some examples, the threshold value may be changed and updated based on patient data. In some examples, the implantable measurement device may be configured to track movement of the patient and determine an angle which the implantable measurement device has moved relative to a vector of Earth's gravity. In other examples, the implantable measurement device is configured to track the direction of movement, which may be analyzed to determine what kind of movement the patient is performing.

Gyroscope data/other sensor data 806 may be data associated with a gyroscope and/or other sensors, such as a load sensor, a temperature sensor, a magnetometer, hall sensors, or a combination thereof. In some examples, the implantable measurement device may only use a gyroscope (described above). In other examples, a gyroscope and one or more additional sensors may be used to collect data.

In some examples, gyroscope data/other sensor data 806 may be used to determine additional movement characteristics and/or confirm that a particular movement (e.g., target activity) is being performed and used to set a threshold value. In some examples, such as when the gyroscope data/other sensor data 806 is gyroscope data, the implantable measurement device may be configured to determine an angle of the implantable measurement device relative to a vector of Earth's gravity. In some examples, such as when the implantable measurement device is located with a knee, a hip, or ankle, when an angle of the implantable measurement device exceeds a certain angle (e.g., threshold angle), the patient may be performing a target activity. In some examples, the may be between 0 degrees and 120 degrees over a sample period. More particularly, the angle may be 10 degrees, 15 degrees, 30 degrees, 45 degrees, 65 degrees or even 90 degrees or more during a sample period. Further, the implantable measurement device may be configured to distinguish between target activities based on the angle of the implantable measurement device associated with the knee joint and/or hip joint. In some examples, when the implantable measurement device is configured as a shoulder or spine implant, when an angle of the implantable measurement device exceeds a certain angle, the patient may be performing a target activity. In one example, when the implantable measurement device is configured as a spine implant, and the angle of the implant exceeds 25 degrees during a sample period, the patient may be bending over, which may be considered a target activity. The determined angle may be different depending on the location of the spine implant in the patient spine. In some examples, when the implant is configured as a spine implant, the implant may measure periods which a patient is bent over. In some examples, an angle of 10 degrees or more may indicate a bending activity. In other examples, a lower threshold angle may be used (e.g. 5 degrees) and may correlate to a loss in posture or time period outside of an optimum posture. In another example, when the implantable measurement device is configured as a shoulder implant, and the angle of the implant exceeds 30 degrees during a sample period, the patient may be reaching out or over head, which may be considered a target activity. In some examples, such as with a shoulder implant, the implant may measure periods of elevation, such as reaching above a patient's head (e.g., reaching into a high cabinet). In this example, a humeral incline of 90 degrees or more during the sample period may indicate an over-head reach. Although some examples described above list particular angles of movement, any angle is contemplated as a threshold angle to determine a target activity. Further, the threshold angle may be adjusted or changed based on each patient and their specific characteristics.

Once the accelerometer data 808 is processed by machine learning algorithm 810, the output from machine learning algorithm 810 updates the accelerometer-based algorithm 812. The accelerometer-based algorithms 812 analyze accelerometer data 808. The output of the accelerometer-based algorithms is a value that is then compared with a threshold value associated with initial classifier 814. In some examples, the machine learning algorithm 810 may also update the threshold value up or down based on patient-specific activity metrics 824 collected by the accelerometer, the gyroscope, one or more additional sensors, or a combination thereof, which will be described further below.

When the output value from the accelerometer-based algorithms 812 is less than the threshold value of initial classifier 814, the patient movement is not performing the target activity. When the patient movement is not the target activity, the implantable measurement device may keep the gyroscope and/or other sensors turned off. In some other examples, when the patient movement is not the target activity, the implantable measurement device may reduce sample rates (i.e., the frequency which the sensor collects data). In some examples, one or more processors of the implantable measurement device may change the sample rate of the first senor (e.g., accelerometer) and/or second sensor (e.g., gyroscope) between 1 Hz and 100 Hz frequencies, depending on battery level, target activity identification, or both. In some examples, a low power sampling frequency may be less than 10 Hz. In other examples, a high power sampling frequency may be between 20 Hz and 100 Hz. In some other examples, the sampling frequency may be less than 1 Hz or greater than 100 Hz. In further examples, when the patient movement is not the target activity, the implantable measurement device may reduce or stop processing signals from the gyroscope and/or other sensors.

When the output value from the accelerometer-based algorithms 812 is greater than the threshold value of initial classifier 814, the patient movement is performing the target activity. When the implantable measurement device has determined that the patient is performing the target activity based on the accelerometer data 808, the gyroscope and/or other sensors may be active 820 to collect data. As discussed above, the other sensors may be a load sensor, a temperature sensor, a magnetometer, or a combination thereof.

When gyroscope/other sensors become active at 820, advanced algorithms 822 may be used to analyze the data to determine an advanced value to be compared with an advanced threshold value. In some examples, an advanced value may be a single value of the combination of the processed accelerometer data 808 and the processed gyroscope/other sensor data 806. In other examples, the advanced value may be a value of the processed gyroscope and/or other sensor data 806. In some examples, the advanced algorithms 822 for analyzing the gyroscope data 606 and accelerometer data 808, as well as any other sensor data, may be revised and updated by machine learning algorithm 810 to change the output of the advanced value relative to the advanced threshold.

The advanced value is then compared with the advanced threshold value of advanced classier 823 to determine whether the patient movement is performing the activity of interest 825 (e.g., target activity). Once the implantable measurement device determines with one or more processors that the patient movement corresponds to the target activity based on accelerometer data 808 and gyroscope/other sensor(s) data, the implantable measurement device records activity metrics 824. Activity metrics 824 are recorded for displaying metrics associated with the target activity for a patient and/or doctor to review to assist with post-operative recovery. Activity metrics 824 may be sent to data library 804 to be saved. Additionally, activity metrics 824 may be received by machine learning algorithm 810 to be processed.

Machine learning algorithm 810 may use activity metrics 824 to update and refine the processing of accelerometer data 808, gyroscope and/or other sensor(s) data 806, accelerometer-based algorithms 812, advanced algorithms 822, initial classifier 814, advanced classifier 823, or a combination thereof. Put differently, the machine learning algorithm 810 may change when the implantable measurement device determines when the patient is performing the target activity by using previously acquired data to update the control process. Similarly, machine learning algorithm 810 may adjust whether each of the sensors is active, the frequency of sampling of each sensor, or both in order to minimize power consumption of the implantable measurement device.

Any reference to implantable measurement device includes any of the measurement devices discussed herein, such as measurement devices 50, 70, 224, 244, 320. Although discussed with reference to implantable measurement devices, the control systems and strategies may be applied with wearable sensors. In some examples, wearable sensors may be used in conjunction with one or more implantable measurement devices to more accurately determine the type of patient movements.

While the present invention has been described with reference to particular embodiments, those skilled in the art will recognize that many changes may be made thereto without departing from the spirit and scope of the present invention. Each of these embodiments and obvious variations thereof is contemplated as falling within the spirit and scope of the invention.

1. A surgical implant, comprising:
an accelerometer;
a sensor;
a processor, the processor configured to:
receive a first data from the accelerometer based on a movement of the surgical implant;
determine a type of movement of the surgical implant based on the first data; and
adjust power to the sensor based on the determined type of movement of the surgical implant.

2. The surgical implant of claim 1, wherein the sensor is powered off during a first movement.

3. The implant of claim 2, wherein the sensor is powered on during a second movement.

4. The surgical implant of claim 1, wherein sensor processing is off during a first movement.

5. The surgical implant of claim 4, wherein sensor processing is on during a second movement.

6. The surgical implant of claim 1, wherein the sensor is multiple sensors.

7. The surgical implant of claim 1, wherein the sensor is at least one of a temperature sensor, a load sensor, and a gyroscope.

8. The surgical implant of claim 1, wherein in a first movement the sensor is deactivated.

9. The surgical implant of claim 8, wherein in a second movement the sensor is activated.

10. The surgical implant of claim 1, wherein the determined type of movement is walking.

11. The surgical implant of claim 1, wherein the determined type of movement is at least one of ascending stairs, descending stairs, changing position between horizontal and vertical, and aerobic activity.

12. The surgical implant of claim 1, wherein the surgical implant is a knee implant.

13. The surgical implant of claim 1, wherein the surgical implant is a shoulder implant.

14. The surgical implant of claim 1, wherein the surgical implant is a spine implant.

15. The surgical implant of claim 1, wherein the surgical implant is a hip implant.

16. A method for measuring movement of an implant via a measurement device, the method comprising:
receiving, via at least one processor, first data from a first sensor, wherein:
the measurement device is coupled to a musculoskeletal system of a patient;
determining a patient movement based on the first data; and
adjusting power supplied to a second sensor based on the determined patient movement.

17-27. (canceled)

28. A method of determining a movement of a patient, the method comprising:
determining a threshold sensor value of a first sensor associated with a first movement type using one or more prior patient datasets;
receiving a first data from a first sensor of an implantable measurement device; and
determining a new threshold sensor value using one or more prior patient data sets and the first data.

29. The method of claim 28, further comprising: activating a second sensor when a first sensor value of the first sensor exceeds the new threshold sensor value.

30. The method of claim 28, wherein the implantable measurement device includes a battery, and the method further comprising:
monitoring a power level of the implantable measurement device.

31. The method of claim 30, further comprising adjusting a sample rate of the first sensor.
